(19) United States
(12) Patent Application Publication
(10) Pub. No.: US 2023/0149489 A1
(43) Pub. Date: May 18, 2023

(54) ANTIBACTERIAL MOUTHRINSE COMPOSITION COMPRISING PROBOSCIDEA JUSSIEUI

(71) Applicants: Saeid Mirzaei, Kerman (IR); Seyedeh Fatemeh Peyro Mousavi, Kerman (IR); Fahimeh Nakhaeipour, Kerman (IR)

(72) Inventors: Saeid Mirzaei, Kerman (IR); Seyedeh Fatemeh Peyro Mousavi, Kerman (IR); Fahimeh Nakhaeipour, Kerman (IR)

(73) Assignees: Saeid Mirzaei, Kerman (IR); Seyedeh Fatemeh Peyro Mousavi, Kerman (IR); Seyedeh Fatemeh Peyro Mousavi, Kerman (IR)

(21) Appl. No.: 18/099,955

(22) Filed: Jan. 22, 2023

Related U.S. Application Data
(60) Provisional application No. 63/302,078, filed on Jan. 23, 2022.

(51) Int. Cl. A61K 36/28; A61K 33/16; A61K 47/26; A61K 47/10; A61Q 11/00

(52) U.S. Cl. CPC A61K 36/28; A61K 33/16; A61K 47/26; A61K 47/10; A61Q 11/00

(57) ABSTRACT

A mouthrinse composition comprising an ethanolic extract of Proboscidea jussieui leaves with a concentration between 0.7% (w/v) and 1.3% (w/v), a fluoride component with a concentration between 0.02% (w/v) and 0.08% (w/v), and propylene glycol with a concentration between 0.7% (w/v) and 1.3% (w/v).

### CROSS-REFERENCE TO RELATED APPLICATION

This application claims the benefit of priority from U.S. Provisional Patent Application Ser. No. 63/302,078, filed on Jan. 23, 2022, entitled “ANTIBACTERIAL MOUTH FORMULA CONTAINING PROBOSCIDEA JUSSIEUI AND PREPARATION METHOD THEREOF” which is incorporated herein by reference in its entirety.

### TECHNICAL FIELD

The present disclosure is generally related to an exemplary mouthrinse composition comprising an exemplary ethanolic extract of Proboscidea jussieui leaves.

### BACKGROUND

Human oral cavity is an ideal habitat for different micro-organisms to develop, and may comprise more than 700 bacterial species. These bacteria colonize on soft and hard tissues and form structured biofilms known as dental plaque. Common oral bacteria may include Porphyromonas gingivalis, Streptococcus mutans, Lactobacillus, and Staphylococcus. A significant bacterial population shift in oral cavity (e.g., due to systemic precondition and/or improper oral hygiene habits) may lead to different oral diseases, such as dental caries, periodontal disease, and gingival disease.

Regular oral hygiene habits like tooth brushing and flossing may effectively remove dental plaque, however disregard of an optimal order of oral hygiene practices may leave behind residual plaque. Thus, taking an additional therapeutic approach, such as mouthrinse compositions, along with mechanical biofilm removal by daily tooth brushing and flossing may result in complete removal of dental plaque. Most of chemical mouthrinse formulations, such as chlorhexidine, are capable of effectively preventing dental plaque and oral diseases. However, long-term and regular use of chemical mouthrinse compositions has been reported to cause different side effects, such as bacterial resistance to common oral antiseptics, cross-resistance between antibiotics and chemical mouthrinse compositions, taste alteration, and teeth stain. Thereby, there is need to alternative oral care mouthrinse compositions derived from natural resources that have been proven to be safe, cost-effective, and effective for restoring normal health.

### SUMMARY

This summary is intended to provide an overview of the subject matter of the present disclosure, and is not intended to identify essential elements or key elements of the subject matter, nor is it intended to be used to determine the scope of the claimed implementations. Its sole purpose is to present some concepts of one or more exemplary aspects in a simplified form as a prelude to the more detailed description that is presented later. The proper scope of the present disclosure may be ascertained from the claims set forth below in view of the detailed description below and the drawings.

One or more exemplary embodiments describe an exemplary mouthrinse composition. In an exemplary embodiment, an exemplary mouthrinse composition may comprise an exemplary ethanolic extract of Proboscidea jussieui leaves, an exemplary fluoride component, and propylene glycol. In an exemplary embodiment, an exemplary ethanolic extract of Proboscidea jussieui leaves has a concentration between about 0.7% (w/v) and 1.3% (w/v). In an exemplary embodiment, an exemplary fluoride component may have a concentration between about 0.02% (w/v) and 0.08% (w/v). In an exemplary embodiment, propylene glycol may have a concentration between about 0.7% (w/v) and 1.3% (w/v).

In an exemplary embodiment, an exemplary fluoride component may comprise sodium fluoride. In an exemplary embodiment, an exemplary mouthrinse composition may further comprise an exemplary sugar alcohol. An exemplary sugar alcohol may comprise Sorbitol with a concentration between about 10% (w/v) and 20% (w/v). In an exemplary embodiment, an exemplary mouthrinse composition may further comprise a flavoring agent. An exemplary flavoring agent may comprise menthol with a concentration between about 0.07% (w/v) and 0.5% (w/v).

This Summary may introduce a number of concepts in a simplified format; the concepts are further disclosed within the “Detailed Description” section. This Summary is not intended to configure essential/key features of the claimed subject matter, nor is intended to limit the scope of the claimed subject matter.

### BRIEF DESCRIPTION OF THE DRAWINGS

The novel features which are believed to be characteristic of the present disclosure, as to its structure, organization, use and method of operation, together with further objectives and advantages thereof, will be better understood from the following drawings in which an exemplary embodiment will now be illustrated by way of example. It is expressly understood, however, that the drawings are for the purpose of illustration and description only and are not intended as a definition of the limits of the present disclosure. Exemplary embodiments will now be described by way of example in association with the accompanying drawings in which:

FIG. 1 illustrates an exemplary flowchart of an exemplary method for producing an exemplary mouthrinse composition, consistent with one or more exemplary embodiments of the present disclosure; and

FIG. 2 illustrates petri plates of Mueller-Hinton agar after inoculation with Streptococcus mutans bacteria, consistent with one or more exemplary embodiments of the present disclosure.

### DETAILED DESCRIPTION

In the following detailed description, numerous specific details are set forth by way of examples to provide a thorough understanding of the relevant teachings related to the exemplary embodiments. However, it should be apparent that the present teachings may be practiced without such details. In other instances, well known methods, procedures, components, and/or circuitry have been described at a relatively high-level, without detail, in order to avoid unnecessarily obscuring aspects of the present teachings.

The following detailed description is presented to enable a person skilled in the art to make and use the methods and devices disclosed in one or more exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be plain to one skilled in the art, and the general principles defined herein may be applied to other implementations and applications without departing from the scope of the present disclosure. The present disclosure is not intended to be limited to the implementations shown, but is to be accorded the widest possible scope consistent with the principles and features disclosed herein.

Disclosed herein is an exemplary mouthrinse composition with antibacterial activity and an exemplary method for producing an exemplary mouthrinse composition. In an exemplary embodiment, an exemplary mouthrinse composition may have antibacterial effect against Enterococcus faecalis and Streptococcus mutans. “antibacterial activity” and/or “antibacterial effect” may refer to any activity including, but not limited to, preventing, inhibiting, and/or reducing growth of bacteria; reducing and/or inhibiting ability of bacteria to produce infection in a subject; reducing and/or inhibiting ability of bacteria to multiply in an environment; and/or reducing and/or inhibiting ability of bacteria to remain infective in an environment. “Antibacterial effect” and/or “antibacterial activity” may also include a decrease in infectivity of bacteria. “Mouthrinse composition,” also referred to as “oral composition” or “mouthwash composition,” may refer to a composition that provides at least one of a therapeutic, prophylactic, and/or cosmetic benefit in oral cavity of a user. Furthermore, “mouthrinse composition” may refer to a product that may not be intentionally swallowed to administer therapeutic agents, but is retained in oral cavity for a time duration to contact with or mucosal tissues and/or tooth surfaces. Tooth surfaces may include, but are not limited to, fissures, pits, occlusal surfaces, crevices, cleft, grooves, irregularities, interstices, depressions, inter-proximal surfaces along the gum line and/or between the teeth, biting or grinding surfaces of a tooth, and/or smooth surfaces of teeth. “Mouthrinse composition” may also refer to a composition capable of curing or treating disease conditions of oral cavity pertaining to plaque, caries, gingivitis, breath malodor, and periodontal related disorders.

In an exemplary embodiment, an exemplary mouthrinse composition may comprise an exemplary ethanolic extract of Proboscidea jussieui leaves. Proboscidea jussieui refers to a plant that has been classified under the family of Martyniaceae, genus of Proboscidea, and species of jussieui. In an exemplary embodiment, an exemplary mouthrinse composition may comprise an exemplary ethanolic extract of Proboscidea jussieui leaves with a concentration between about 0.7% (w/v) and 1.3% (w/v). In an exemplary embodiment, an exemplary mouthrinse composition may comprise an exemplary ethanolic extract of Proboscidea jussieui leaves with a concentration of about 1% (w/v). “Extract” may refer to a phytochemical material obtained from a plant material/segment by an extraction process (using an extraction solvent). For example, “ethanolic extract” may refer to a phytochemical material extracted from a plant material using pure ethanol (e.g., a 99% or 100% (v/v) ethanol) or ethanol-water solution (e.g., 70% (v/v) ethanol) as an extraction solvent.

In an exemplary embodiment, an exemplary mouthrinse composition may further comprise an exemplary fluoride component and propylene glycol. In an exemplary embodiment, an exemplary mouthrinse composition may comprise an exemplary fluoride component with a concentration between about 0.02% (w/v) and 0.08% (w/v). In an exemplary embodiment, an exemplary mouthrinse composition may comprise propylene glycol with a concentration between about 0.7% (w/v) and 1.3% (w/v). In an exemplary embodiment, an exemplary mouthrinse composition may comprise about 1% (w/v) of an exemplary ethanolic extract of Proboscidea jussieui leaves, about 0.05% (w/v) of an exemplary fluoride component, and about 1% (w/v) propylene glycol. In an exemplary embodiment, an exemplary fluoride component may include, but is not limited to, inorganic fluoride salts, such as soluble alkali metal, alkaline earth metal salts such as potassium fluoride, sodium fluoride, calcium fluoride, ammonium fluoride, cuprous fluoride, copper fluoride, zinc fluoride, sodium fluorosilicate, ammonium fluorosilicate, barium fluoride, sodium fluorozirconate, sodium monofluorophosphate, ammonium fluorozirconate, fluorinated sodium calcium pyrophosphate, aluminum mono and/or di-fluorophosphate, tin fluorides, alkali metals, stannous fluorides, sodium fluorides, sodium monofluorophosphate, and a combination thereof. In an exemplary embodiment, an exemplary mouthrinse composition may comprise sodium fluoride with a concentration between about 0.02% (w/v) and 0.08% (w/v).

In an exemplary embodiment, an exemplary mouthrinse composition may further comprise a sweetening agent including, but not limited to, artificial, natural, and water-soluble sweeteners, dipeptide-based sweeteners, and/or water-soluble artificial sweeteners. In an exemplary embodiment, an exemplary mouthrinse composition may comprise an exemplary water-soluble sweetener, such as monosaccharides, disaccharides, and polysaccharides such as ribose, xylose, mannose, glucose, galactose, dextrose, fructose, sucrose, maltose, partially hydrolyzed starch or corn syrup solids and sugar alcohols such as sorbitol, mannitol, xylitol, and hydrogenated glucose syrup. In an exemplary embodiment, an exemplary mouthrinse composition may comprise an exemplary water-soluble artificial sweetener, such as exemplary soluble saccharin salts (e.g., calcium or sodium saccharin salts), cyclamate salts (e.g., sodium salt), and free acid form of saccharin. In an exemplary embodiment, an exemplary mouthrinse composition may comprise an exemplary dipeptide-based sweetening agent, such as dihydrochalcone, L-aspartyl-L-phenyl-alanine methyl ester, glycyrrhizin, and/or Stevia rebaudiana.

In an exemplary embodiment, an exemplary mouthrinse composition may comprise an exemplary synthetic sweetener, such as 3,6-dihydro-6-methyl-1,2,3-oxathiazin-4-one-2,2-dioxide, potassium salts of 3,6-dihydro-6-methyl-1,2,3-oxathiazin-4-one-2,2-dioxide, sodium salts of 3,6-dihydro-6-methyl-1,2,3-oxathiazin-4-one-2,2-dioxide, and calcium salts of 3,6-dihydro-6-methyl-1,2,3-oxathiazin-4-one-2,2-dioxide. The amount of an exemplary sweetener may vary based on the type of sweetener used for producing an exemplary mouthrinse composition. In an exemplary embodiment, an exemplary mouthrinse composition may comprise an exemplary water-soluble sweetener derived from naturally occurring water-soluble sweeteners, such as an exemplary chlorinated derivative of sucrose and exemplary protein-based sweeteners (e.g., thaumatococus danielli (Thaumatin I and II)). In an exemplary embodiment, an exemplary mouthrinse composition may comprise an exemplary sweetening agent with a concentration between about 0.005% (w/w) and 5%. In an exemplary embodiment, an exemplary mouthrinse composition may comprise Sorbitol with a concentration between about 10% (w/v) and 20% (w/v). In an exemplary embodiment, an exemplary mouthrinse composition may comprise Sorbitol with a concentration of about 15% (w/v).

In an exemplary embodiment, an exemplary mouthrinse composition may further comprise an exemplary flavoring agent. An exemplary flavoring agent may include, but is not limited to, thymol, menthol, methyl salicylate, cinnamon, eucalyptol, spearmint, peppermint, and/or wintergreen. In an exemplary embodiment, an exemplary mouthrinse composition may comprise menthol with a concentration between about 0.07% (w/v) and 0.5% (w/v). In an exemplary embodiment, an exemplary mouthrinse composition may comprise menthol with a concentration of about 0.1% (w/v).

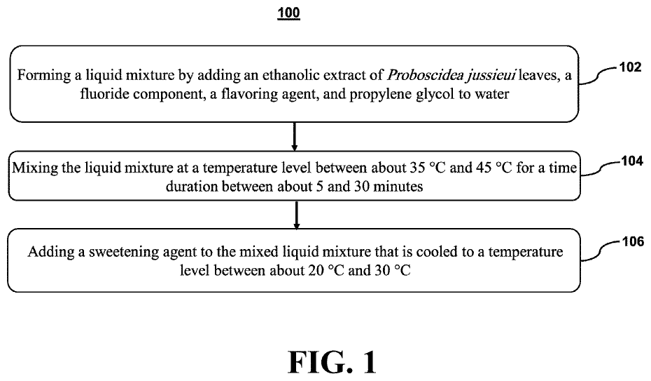

Referring to the figures, FIG. 1 illustrates an exemplary flowchart of exemplary method 100 for producing an exemplary mouthrinse composition, consistent with one or more exemplary embodiments of the present disclosure. In an exemplary embodiment, exemplary method 100 may include: forming an exemplary liquid mixture by adding an exemplary ethanolic extract of Proboscidea jussieui leaves, an exemplary fluoride component, an exemplary flavoring agent, and propylene glycol to water (step 102), mixing an exemplary liquid mixture at a temperature level between about 35° C. and 45° C. for a time duration between about 5 and 30 minutes (step 104), and adding an exemplary sweetening agent to an exemplary mixed liquid mixture that is cooled to a temperature level between about 20° C. and 30° C. (step 106).

In further detail with respect to step 102, step 102 may include forming an exemplary liquid mixture by adding an exemplary ethanolic extract of Proboscidea jussieui leaves, an exemplary fluoride component, an exemplary flavoring agent, and propylene glycol to water. In an exemplary embodiment, an exemplary ethanolic extract of Proboscidea jussieui leaves may be formed through an exemplary ethanol extraction process. In an exemplary embodiment, an exemplary ethanol extraction process may include adding an exemplary dried powder of Proboscidea jussieui leaves with a final concentration between about 5% (w/v) and 15% (w/v) to an ethanol solution (with a concentration of at least 96% (v/v)) in an exemplary laboratory container (e.g., beaker, flask, etc.). In an exemplary embodiment, an exemplary ethanol extraction process may further include shaking/mixing an exemplary mixture of Proboscidea jussieui leaves powder and ethanol at about 22-30° C. for a time duration between about 30 and 80 hours, using a stirrer or agitation machine (e.g., a magnetic stirrer).

In an exemplary embodiment, an exemplary ethanol extraction process may further include vacuum filtering an exemplary shaken mixture of Proboscidea jussieui leaves powder and ethanol through an exemplary cellulose filter paper to remove larger plant particles followed by concentrating an exemplary filtered mixture by rotary evaporation (rotary evaporation may result in the evaporation of ethanol). For example, forming about 50-100 mL of an exemplary ethanolic extract of Proboscidea jussieui leaves may include: obtaining about 100-300 g Proboscidea jussieui leaves powder by grinding Proboscidea jussieui leaves (i.e., dried leaves) in an exemplary laboratory plate (e.g., using a pestle); adding an exemplary powder of Proboscidea jussieui leaves with a final concentration between about 5% (w/v) and 15% (w/v) to ethanol 96% (v/v) in an exemplary laboratory container (e.g., beaker, flask, etc.); shaking an exemplary mixture of Proboscidea jussieui leaves powder and ethanol at about 23-27° C. for a time duration between about 30 and 80 hours, using a stirrer or agitation machine (e.g., a magnetic stirrer); vacuum filtering an exemplary shaken mixture of Proboscidea jussieui leaves powder and ethanol through an exemplary cellulose filter paper; evaporating ethanol from an exemplary filtered mixture by rotary evaporation until obtaining about 50-100 mL of an exemplary ethanolic extract of Proboscidea jussieui leaves.

In further detail with respect to step 102, forming an exemplary liquid mixture by adding an exemplary ethanolic extract of Proboscidea jussieui leaves, an exemplary fluoride component, an exemplary flavoring agent, and propylene glycol to water may include adding an exemplary ethanolic extract of Proboscidea jussieui leaves (with a final concentration between about 0.7% (w/v) and 1.3% (w/v)), an exemplary fluoride component (with a final concentration between about 0.02% (w/v) and 0.08% (w/v)), an exemplary flavoring agent (with a final concentration between about 0.07% (w/v) and 0.5% (w/v)), and propylene glycol (with a final concentration between about 0.7% (w/v) and 1.3% (w/v)) to a predetermined amount of water, in an exemplary laboratory container. An exemplary laboratory container may include, but is not limited to, beakers, tins, flasks, bottles, buckets, basins, bowls, vials, tubes, barrels, cannisters, etc. In an exemplary embodiment, an exemplary water used for forming an exemplary liquid mixture may include distilled water, double-distilled water, or ultrapure water. “Ultrapure water” may refer to a water that has been purified using a combination of ultrafiltration technologies and ultraviolet photo-oxidation system. Amount of water may be determined based on the amount of an exemplary mouthrinse composition that is intended to be produced.

In an exemplary embodiment, adding an exemplary ethanolic extract of Proboscidea jussieui leaves, an exemplary fluoride component, an exemplary flavoring agent, and propylene glycol to water may include adding an exemplary ethanolic extract of Proboscidea jussieui leaves (with a final concentration between about 0.7% (w/v) and 1.3% (w/v)), sodium fluoride (with a final concentration between about 0.02% (w/v) and 0.08% (w/v)), menthol (with a final concentration between about 0.07% (w/v) and 0.5% (w/v)), and propylene glycol (with a final concentration between about 0.7% (w/v) and 1.3% (w/v)) to a predetermined amount of deionized water, in an exemplary laboratory container. For example, preparing about 200 mL of an exemplary mouthrinse composition may include adding about 2 g of an exemplary ethanolic extract of Proboscidea jussieui leaves, about 100 mg sodium fluoride, about 200 mg menthol, and about 2 g propylene glycol to about 200 mL deionized water, in an exemplary laboratory container.

In further detail with respect to step 104, step 104 may include mixing an exemplary liquid mixture at a temperature level between about 35° C. and 45° C. for a time duration between about 5 and 30 minutes. In an exemplary embodiment, mixing an exemplary liquid mixture at a temperature level between about 35° C. and 45° C. for a time duration between about 5 and 30 minutes may include stirring an exemplary liquid mixture, using a stirrer (e.g., a magnetic stirrer or an agitation machine), for a time duration between about 5 and 30 minutes such that the temperature level of an exemplary liquid mixture is maintained at a temperature level between about 35° C. and 45° C. during stirring. In an exemplary embodiment, mixing an exemplary liquid mixture at a temperature level between about 35° C. and 45° C. for a time duration between about 5 and 30 minutes may include stirring an exemplary liquid mixture at a temperature level of about 40° C. for a time duration of about 10 minutes to obtain a homogenous and optically clear mouthwash composition.

In further detail with respect to step 106, step 106 may include adding an exemplary sweetening agent to an exemplary mixed liquid mixture that is cooled to a temperature level between about 20° C. and 30° C. In an exemplary embodiment, adding an exemplary sweetening agent to an exemplary mixed liquid mixture that is cooled to a temperature level between about 20° C. and 30° C. may include adding an exemplary sweetening agent to an exemplary mixed liquid mixture that is cooled to a temperature level between about 23° C. and 27° C. (i.e., room temperature) by placing an exemplary mixed liquid mixture in a place/room with a temperature of about 23-27° C. In an exemplary embodiment, adding an exemplary sweetening agent to an exemplary mixed liquid mixture that is cooled to a temperature level between about 20° C. and 30° C. may include adding Sorbitol to an exemplary mixed liquid mixture that is cooled to a temperature level between about 23° C. and 27° C. (i.e., room temperature).

In an exemplary embodiment, an exemplary mouthwash composition may be formulated in different deliverable forms including, but not limited to, oral disinfecting solids, oral disinfecting liquids, solid dosage forms, oral gargling compositions, controlled release forms, and/or sustained release forms. In an exemplary embodiment, an exemplary mouthrinse composition may further include one or more exemplary additives including, but not limited to, thickeners, flavors, defoamers, sweeteners, humectants, buffers, softeners, astringents, and a combination thereof.

### EXAMPLES

Hereinafter, one or more exemplary embodiments will be described in further detail with reference to examples. It will be obvious to a person having ordinary skill in the art that these examples may be for illustrative purposes only and are not to be interpreted to limit the scope of the present disclosure.

### EXAMPLE 1: Preparing Alcoholic Extract of Proboscidea jussieui

In this example, an exemplary ethanolic extract of Proboscidea jussieui leaves was prepared using an exemplary alcoholic extraction method similar to an exemplary method for preparing an exemplary ethanolic extract for steps of exemplary method 100. Proboscidea jussieui leaves were dried under shade drying conditions. In an exemplary embodiment, drying under shade drying conditions may refer to drying Proboscidea jussieui leaves in a ventilated room with a temperature between about 23° C. and 27° C. for about 3-10 days. Exemplary dried leaves were grounded in a mechanical or electric blender.

About 200 g of Proboscidea jussieui leaves powder was mixed with about 2 L of ethanol 96% (v/v) in an exemplary laboratory container. Then, an exemplary mixture of Proboscidea jussieui leaves powder and ethanol was shaken (using a magnetic stirrer or an agitation machine) for about 72 hours in a room with a temperature of about 23-27° C. The shaken mixture of Proboscidea jussieui leaves powder and ethanol was then filtered through an exemplary cellulose filter paper (e.g., using a vacuum filtration system). Subsequently, ethanolic solvent of an exemplary filtered mixture was evaporated to dryness (e.g., until obtaining about 50 mL extract) using a rotary evaporator at a temperature level below 50° C.

### EXAMPLE 2: Preparing Mouthrinse Composition Comprising Proboscideajussieui Leaves Extract

In this example, an exemplary mouthrinse composition (i.e., liquid composition) comprising an exemplary ethanolic extract of Proboscidea jussieui leaves was prepared based on a method similar to exemplary method 100. In an exemplary embodiment, to prepare about 200 mL of an exemplary mouthrinse composition, about 2 g of ethanolic extract of Proboscidea jussieui leaves, about 100 mg sodium fluoride, about 200 mg menthol, about 3 g phosphoric acid, and about 2 g propylene glycol were weighed and added to about 200 mL deionized water (in an exemplary laboratory container, such as a beaker) and was stirred at about 40° C. for a time duration of about 10 minutes using a magnetic stirrer.

An exemplary stirred liquid mixture was then cooled to a temperature level between about 23° C. and 27° C. (i.e., room temperature) by placing an exemplary stirred liquid mixture in a room with a temperature of about 23-27° C. Subsequently, about 30 g Sorbitol was added to an exemplary cooled liquid mixture and stirred at 23-27° C. for a time duration of about 3-30 minutes (using a magnetic stirrer) to obtain a homogenous and optically clear mouthwash composition.

### EXAMPLE 3: Determination of Minimum Inhibitory and Bactericidal Concentration of Mouthrinse Composition Against Streptococcus mutans

In this example, the minimum inhibitory concentration (MIC) of an exemplary mouthrinse composition against Streptococcus mutans (S. mutans) was determined based on an exemplary broth microdilution method using a 96-well microtiter plate.

Exemplary bacterial suspensions were prepared by inoculating S. mutans bacteria in an exemplary saline solution (e.g., 0.85% (w/v) NaCl) followed by adjusting the inoculated solution to an exemplary turbidity of about 0.5 McFarland (i.e., 2×10⁸ colony forming units (CFU)/μL). Subsequently, exemplary bacterial suspensions were diluted in a cation-adjusted Mueller Hinton-II broth with a volume ratio (bacterial suspension: cation-adjusted Mueller Hinton-II broth) of about 1:100. About 100 μL of Brain-Heart Infusion (BHI) medium containing about 1.5×10⁶ CFU/mL of S. mutans was added to each well of an exemplary 96-well plate. Then, 50 μL of exemplary dilutions of an exemplary mouthrinse composition were added to each well of the 96-well plate. In an exemplary implementation, an exemplary BHI medium containing bacterial suspension of S. mutans (without an exemplary mouthrinse composition) was added to one or more wells as an exemplary positive control. Meanwhile, an exemplary BHI medium containing an exemplary mouthrinse composition (without bacterial suspension of S. mutans) was added to one or more wells as an exemplary negative control. In an exemplary row of the 96-well plate, exemplary dilutions of 0.2% (w/v) chlorhexidine mixed with the inoculated broth of Mueller Hinton-II were added to each well, as an exemplary standard sample.

The microtiter plate was further incubated for 24 hours in an incubator with a temperature of about 37° C. After 24 hours of culture, bacterial growth was determined by measuring the light absorbance of each well at 600 nm (OD₆₀₀) using an ELISA reader. The MIC value may be defined as the lowest concentration of an exemplary mouthrinse composition that may prevent bacterial growth. To distinguish which well of the 96-well plate comprises an MIC of an exemplary mouthrinse composition against S. mutans, exemplary suspensions of the wells having a turbidity similar to negative control wells were diluted in an autoclaved physiological serum with a volume ratio (exemplary suspension: physiological serum) of about 1:1000. Then, about 100 μL of the diluted suspensions were poured and distributed on Mueller-Hinton agar and incubated for 24 hours in an incubator with a temperature of about 37° C. Grown colonies were counted after 24 hours of incubation.

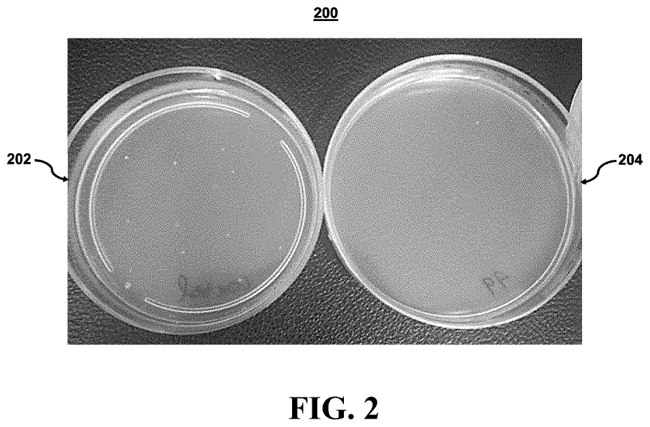

FIG. 2 illustrates petri plates of Mueller-Hinton agar 200 after inoculation with S. mutans bacteria, consistent with one or more exemplary embodiments of the present disclosure. In further detail with respect to petri plates of Mueller-Hinton agar 200, plate 202 contains an exemplary Mueller-Hinton agar inoculated with the suspension of S. mutans and an exemplary mouthrinse composition. Plate 204 is an exemplary control plate of Mueller-Hinton agar that was inoculated with S. mutans bacteria without being mixed with an exemplary mouthrinse composition. Thus, MIC value of an exemplary mouthrinse composition against S. mutans equals to the concentration of an exemplary mouthrinse composition in the well that was selected to be inoculated on Mueller-Hinton agar of plate 202. Thereby, MIC of an exemplary mouthrinse composition was measured to be about 0.125% (w/v). Comparing the number of grown colonies in plate 202 and plate 204, the minimum bactericidal concentration (MBC) of an exemplary mouthrinse composition was calculated to be about 0.25% (w/v). MBC is defined as the minimum concentration of a substance (e.g., an exemplary mouthrinse composition) that kills 99.9% of the tested bacteria in a culture medium.

### EXAMPLE 4: Determination of Antimicrobial Activity of an Exemplary Mouthrinse Composition by Agar-Well Diffusion Method

In this example, the antibacterial activity of an exemplary mouthrinse composition against Enterococcus faecalis (E. faecalis) was evaluated by agar-well diffusion technique. The surface of an exemplary agar plate was inoculated with an exemplary inoculum of E. faecalis. Then, agar was cut using sterile cork borer to create 6×6 mm² wells. An exemplary empty well in a first petri plate was filled with about 20-100 μL of an exemplary mouthrinse composition comprising about 1% (w/v) of an exemplary ethanolic extract of Proboscidea jussieui leaves, and an exemplary empty well in a second petri plate was filled with chlorhexidine 0.2% (w/v) as an exemplary standard sample. Subsequently, petri plates were placed in an incubator with a temperature level of about 37° C. and incubated for about 24 hours. After 24 hours, inhibition zones—that were formed in result of diffusion of an exemplary mouthrinse composition in agar medium and, in turn, inhibition of E. faecalis growth—were measured. The obtained results in this example revealed an inhibition zone of about 15 mm and about 16 mm for an exemplary mouthrinse composition and chlorhexidine 0.2% (w/v), respectively.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. An element proceeded by “a” or “an” does not, without further constraints, preclude the existence of additional identical elements in the process, method, article, or apparatus that comprises the element.

Unless otherwise stated, all measurements, values, ratings, positions, magnitudes, sizes, and other specifications that are set forth in this specification, are approximate, not exact. They are intended to have a reasonable range that is consistent with the functions to which they relate and with what is customary in the art to which they pertain.

It will be understood that the terms and expressions used herein have the ordinary meaning as is accorded to such terms and expressions with respect to their corresponding respective areas of inquiry and study, except where specific meanings have otherwise been set forth herein. Relational terms such as “first” and “second” and the like may be used solely to distinguish one entity or action from another without necessarily requiring or implying any actual such relationship or order between such entities or actions.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it may be seen that various features are grouped together in various implementations. This is for purposes of streamlining the disclosure, and is not to be interpreted as reflecting an intention that the claimed implementations require more features than are expressly recited in each claim. Rather, as the following claims reflect, inventive subject matter lies in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in light of the attached claims and their equivalents. Also, various modifications and changes may be made within the scope of the attached claims.

What is claimed is:

1. A mouthrinse composition, comprising:
an ethanolic extract of Proboscidea jussieui leaves with a concentration between 0.7% (w/v) and 1.3% (w/v);
a fluoride component with a concentration between 0.02% (w/v) and 0.08% (w/v); and
propylene glycol with a concentration between 0.7% (w/v) and 1.3% (w/v).

2. The mouthrinse composition of claim 1, wherein the ethanolic extract of Proboscidea jussieui leaves with the concentration between 0.7% (w/v) and 1.3% (w/v) comprises the ethanolic extract of Proboscidea jussieui leaves with a concentration of 1% (w/v).

3. The mouthrinse composition of claim 1, wherein the fluoride component comprises sodium fluoride.

4. The mouthrinse composition of claim 1, further comprising a sugar alcohol.

5. The mouthrinse composition of claim 4, wherein the sugar alcohol comprises Sorbitol with a concentration between 10% (w/v) and 20% (w/v).

6. The mouthrinse composition of claim 1, further comprising a flavoring agent.

7. The mouthrinse composition of claim 6, wherein the flavoring agent comprises menthol.

8. The mouthrinse composition of claim 7, wherein the menthol has a concentration between 0.07% (w/v) and 0.5% (w/v).
